(19) United States
(12) Patent Application Publication
(10) Pub. No.: US 2024/0233930 A1
(43) Pub. Date: Jul. 11, 2024

(54) SYSTEM AND METHOD FOR A MEDICAL REFERRAL OR DISEMBARK BETWEEN A MARITIME VESSEL AND SHORESIDE ENTITIES

(71) Applicant: Tritan Software Corporation, Miami, FL (US)

(72) Inventors: Andrew L. Carricarte, Miami, FL (US); Abel Jose Almeida, Miami, FL (US)

(73) Assignee: Tritan Software Corporation, Miami, FL (US)

(21) Appl. No.: 18/615,554

(22) Filed: Mar. 25, 2024

Related U.S. Application Data
(63) Continuation of application No. 16/730,410, filed on Dec. 30, 2019.

(51) Int. Cl. G16H 40/20; B63B 27/00; G06Q 50/26; G16H 10/60; G16H 80/00; H04L 9/40

(52) U.S. Cl. CPC G16H 40/20; B63B 27/00; G06Q 50/26; G16H 10/60; G16H 80/00; H04L 63/04

(57) ABSTRACT

A method of facilitating patient care between a maritime vessel movable on a body of water and at least one shoreside medical provider. The method includes upon determination that a patient on board the maritime vessel requires medical attention, determining whether the patient should be referred to or medically disembarked to the at least one shoreside medical provider, identifying a target shoreside medical provider from the at least one shoreside medical provider qualified to care for the patient, and coordinating care of the patient by the identified target shoreside medical provider, the coordination of care including coordinating exchange of any necessary data between the maritime vessel movable on a body of water and at least one of the identified target shoreside medical provider, one or more third party servicers, one or more shoreside vessel management entities, and one or more governing agencies, in order to facilitate care of the patient.

### CROSS-REFERENCE TO RELATED APPLICATION

This application is a Continuation of U.S. Non-Provisional patent application Ser. No. 16/730,410, filed on Dec. 30, 2019, which is incorporated herein by reference in its entirety.

### TECHNICAL FIELD

The present disclosure relates to patient referral systems and more specifically to a method and system for referring a patient onboard a maritime vessel who becomes ill or injured to an appropriate offshore medical provider based on a variety of variables either with or without the assistance of the offshore medical provider and/or a third party.

The present disclosure relates to patient referral systems and more specifically to a method and system for referring a patient onboard a maritime vessel who becomes ill or injured to an appropriate offshore medical provider based on a variety of variables either with or without the assistance of the offshore medical provider and/or a third party.

### BACKGROUND

The maritime industry has millions of contractors, crew and guest passengers that embark on voyages each year for both commercial and leisure purposes. These vessels are both stationary and/or travel distances through and to various geographic territories and destinations around the world. Due to the nature and volume of this type of travel, the occurrence of medical events for crew and guest passengers will inevitably occur when the vessel is off shore. This requires either a short-term medical referral or a longer-term disembark in order for the patient to receive medical treatment. It will also require coordination between the vessel, governing agencies, e.g., national governments, port authorities, public health departments, etc., and the shoreside medical provider(s), which may include, for example, coordination with third party servicer(s) and/or the shoreside vessel management entity or entities.

There are significant challenges in trying to coordinate medical care for a patient on a maritime vessel while the vessel is stationary or is travelling across bodies of water to various domestic or international destinations and ports. For example, often there is a lack of connectivity or means to exchange information and communication with the vessel and the shoreside medical provider, resulting in the inability to coordinate for proper care. There is also often a lack of ability to coordinate with appropriate parties or governing agencies for activities such as transportation, medical coordination, port clearance, reporting, etc., which often requires assistance from third-party servicer(s), the shoreside vessel management entity or entities, and/or governing agencies, thus resulting in delays in treatment and risks to the patient. Further, there is often an inability to exchange important medical information securely and in a compliant manner resulting in phone calls or unsecure emails due to the complex environments on a vessel that often times do not have or support standard software systems. Another challenge is the lack of tools for which to guide onboard staff as to when they should seek care or what is available at different destinations and routes resulting in significant risk to the crew or passenger safety and life.

Many times, onboard staff are ill-equipped or untrained to address these issues, unfamiliar with the availability or options for shore side care and international protocol across various destinations, or lack available information as to where to send the crew or passenger or with whom to coordinate for medical care.

The present disclosure provides a method and system that addresses the above-described challenges.

### SUMMARY

In accordance with one aspect of the present disclosure, a method of facilitating patient care between a maritime vessel movable on a body of water and at least one shoreside medical provider, is provided. The method includes upon determination that a patient on board the maritime vessel requires medical attention, determining whether the patient should be referred to or medically disembarked to the at least one shoreside medical provider, identifying a target shoreside medical provider from the at least one shore side provider qualified to care for the patient, and coordinating care of the patient by the identified target shoreside medical provider, the coordination of care including coordinating exchange of any necessary data between the maritime vessel movable on a body of water and at least one of the identified target shoreside medical provider, one or more third party servicers, one or more shoreside vessel management entities, and one or more governing agencies, in order to facilitate the care of the patient.

According to one embodiment of this aspect, the determining whether the patient should be referred to or medically disembarked to the at least one shore side medical provider includes consideration of at least one of patient medical history, patient personal information, patient's vitals, primary medical complaint, diagnosis, patient's physical examination results, results of onboard investigation, availability of medical care of qualified personnel onboard vessel, severity of patient's condition, patient consent, and geographic location of maritime vessel. According to another embodiment, identifying a target shoreside medical provider from the at least one shoreside medical provider qualified to care for the patient comprises consideration of at least one of location of the maritime vessel, at least one of proximity and access of the maritime vessel to the at least one shore side medical provider, severity of the patient's condition, diagnosis, medical specialty required, contractual arrangements, quality of medical care at the at least one shore side medical provider, customs of the country where the patient is to disembark, and costs.

According to another embodiment, coordinating the exchange of the necessary data includes consideration of at least one of, the patient's medical information, patient's status. scheduling information, costs, employment information, payment information, transportation information, governing agency requirements, consent, and fit-for-duty status prior to, during, or after the referral or medical disembark. According to another embodiment, the exchange of necessary data occurs via a secure communication channel. According to another embodiment, after care is provided by the target shoreside medical provider, the method further includes determining if the patient is to continue treatment with the shoreside medical provider, is to be repatriated, or is to be transported to another shoreside medical provider. According to another embodiment, where determining if the patient is to continue treatment with the shoreside medical provider, is to be repatriated, or is to be transported to another shoreside medical provider depends at least upon the patient's fitness for duty, the patient's fitness to sail status, medical needs, medical availability, employment status, alternatives for care within the patient's home country, patient or family consent, a calculated maximum medical improvement (MMI) status for the patient, and contractual or legal requirements. According to another embodiment, the method further includes assigning the patient to the vessel if the patient is fit for duty or fit to sail, and updating the patient record after care has been provided.

In accordance with another aspect of the present disclosure, a system for facilitating patient care between a maritime vessel situated offshore and at least one shoreside entity, is provided. The system includes processing circuitry including a memory and a processor, the memory in communication with the processor, the memory having instructions that, when executed by the processor, configure the processor to, upon determination that a patient on board the maritime vessel requires medical attention, determine whether the patient should be referred to or medically disembarked to the at least one shoreside medical provider, identify a target shoreside medical provider from the at least one shore side medical provider qualified to care for the patient, and coordinate care of the patient by the identified target shoreside medical provider, the coordination of care including coordinating exchange of any necessary data between the maritime vessel movable on a body of water and at least one of the identified target shoreside provider, one or more third party servicers, one or more shoreside vessel management entities, and one or more governing agencies, in order to facilitate the care of the patient.

According to one embodiment of this aspect, determining whether the patient should be referred to or medically disembarked to the at least one shore side medical provider comprises consideration of at least one of patient medical history, patient personal information, patient vitals, primary medical complaint, diagnosis, patient physical examination results, results of onboard investigation or diagnostics, availability of medical care of qualified personnel onboard the maritime vessel, severity of patient's condition, patient consent, geographic location of the maritime vessel and impact on itinerary or operations.

According to another embodiment, identifying a target shoreside medical provider from the at least one shoreside medical provider qualified to care for the patient comprises consideration of at least one of location of the maritime vessel, at least one of proximity and access of the maritime vessel to the at least one shoreside medical provider, severity of the patient's condition, diagnosis, medical specialty required, contractual arrangements, quality of medical care at the at least one shoreside medical provider, customs of the country where the patient is to disembark, and costs. According to another embodiment, coordinating the exchange of the necessary data includes consideration of at least one of, the patient's medical information, patient's status, scheduling information, costs, employment information, payment information, transportation information, governing agency requirements, consent, and fit-for-duty status, fit to sail status, prior to, during, or after the referral or medical disembark. According to another embodiment, the exchange of necessary data occurs via a secure communication channel.

According to another embodiment, wherein after care is provided by the target shoreside medical provider, the method further includes determining if the patient is to continue treatment with the shoreside medical provider, is to be repatriated, or is to be transported to another shoreside medical provider. According to another embodiment, where determining if the patient is to continue treatment with the shoreside medical provider, is to be repatriated, or is to be transported to another shoreside medical provider depends at least upon the patient's fitness for duty, the patient's fitness to sail status, medical needs, medical availability, employment status, alternatives for care within the patient's home country, patient or family consent, a calculated maximum medical improvement (MMI) status for the patient, and contractual or legal requirements. According to another embodiment, the processor is further configured to assign the patient to the vessel if the patient is fit for duty, or fit to sail, and update the patient record after care has been provided.

### BRIEF DESCRIPTION OF DRAWINGS

FIG. 1 is an illustration of the computer system of the present disclosure;

FIG. 2 is a flowchart showing steps taken by the onboard system of the user in accordance with the principles of the present disclosure;

FIG. 3 is a flowchart showing steps taken by the user's shoreside system in accordance with the principles of the present disclosure;

FIG. 4 is a flowchart showing steps taken by a shoreside third party servicer in accordance with the principles of the present disclosure;

FIG. 5 is a flowchart showing steps taken by an external system of the third party servicer in accordance with the principles of the present disclosure;

FIG. 6 is a flowchart showing steps taken by the system of the shoreside medical provider in accordance with the principles of the present disclosure; and

FIG. 7 is a flowchart showing steps taken by an external system of the shoreside medical provider in accordance with the principles of the present disclosure.

### DETAILED DESCRIPTION OF EXEMPLARY EMBODIMENTS

In an exemplary embodiment, the present disclosure provides a system and method, connected via satellite, internet or other means of electronic communication, configured to provide the ability for a maritime vessel operating on a body of water, to determine and coordinate a medical referral or disembark of a person onboard to a medical provider operating on land with the assistance of a user on a vessel and/or shoreside vessel management entity and/or a third party servicer facilitating coordination to that shoreside medical provider.

Throughout this disclosure, the term “maritime vessel” or “vessel” shall be defined as cargo ships, passenger transport, cruise ships, leisure/luxury craft (yachts), offshore platforms/rigs, support vessels, tankers, liquefied natural gas (LNG) transport, etc.

Throughout this disclosure, the term “shoreside medical provider” or “medical provider” shall be defined as an individual or entity that provides medical care or medical services for sick or injured people.

Throughout this disclosure, the term “shoreside vessel management entity” or “vessel management entity” shall be defined as an individual or entity that may own, manage, service and/or partially or fully operate a single or multiple fleet of vessels.

Throughout this disclosure, the term “third party servicer” shall be defined as an individual or entity that provides services on behalf of the shoreside vessel management entity to provision and/or facilitate the coordination of medical care and/or medical services. It is noted that the third party servicer can, in some embodiments, also act and perform the functions of the shoreside medical provider and the vessel management entity and vice versa.

Throughout this disclosure, the term “referral” or “medical referral” shall be defined as referrals from onboard staff with our without a third party servicer and/or the shoreside vessel management entity to a shoreside medical provider for an onboard crew or passenger typically with the expectation that they will return back to the vessel prior to embarkation usually occurring on the same day.

Throughout this disclosure, the term “disembark” or “medical disembark” shall be defined as more elaborate treatment requirements under which a crew member or passenger will go through the process of formally disembarking a ship (i.e., clearing port authorities, clearing customs, finalizing clearing procedures onboard, etc.) with the intent to receive medical treatment typically with the expectation that they will not return back to the ship or duty prior to embarkation.

Throughout this disclosure, the term “patient” shall mean a crew member, guest, contractor, or passenger aboard a vessel on a body of water.

The system of the present disclosure has one or more software applications operating on the same technical ecosystem that allow for full or limited access by each party in order to coordinate and collaborate the coordination. One such software application onboard has the ability to operate over internet or offline operation and synchronize via a variety of communication protocols including, for example, satellite connectivity, cellular/data networks, etc. The onboard application is designed to operate in a limited connectivity environment while being connected and synchronized with the other applications of the system. This provides the other applications operating on the same system with the most current and critical information required to manage and coordinate care.

Other software applications in the system include those under which shoreside entities (i.e., one or more shoreside vessel management entities, one or more third party servicers, one or more governing agencies, etc.) and shoreside medical providers can also access and collaborate simultaneously or independently with the information from onboard via secure online or offline connection. These represent software applications with roles and privileges intended to distinctly serve these shoreside entities such as, for example, an insurance company, a medical assistance company, a port agent or authority, a government immigration or customs authority, health departments (e.g., medical transportation company), standard transportation company, medical clinic or hospital, and/or an overseeing organizational, management, and/or operational body.

Thus, the present disclosure is a computer-based system that solves the problems associated with coordinating how a patient that falls ill or is injured on a vessel on a body of water can be disembarked and referred to a selected shoreside medical provider, how to coordinate the transportation of the disembarked patient to the selected shoreside medical provider, and how to arrange for communication among the parties involved, including access to and transmission of the patient's medical records, approval and appointment requests, and the updating of the patient's medical records by both the shoreside medical provider that treated the patient and/or the shoreside entities (i.e., shoreside vessel management entity or entities, one or more third party servicers, one or more governing agencies, etc.) and the vessel, should the patient return to it. Note that the patient's medical “record” as referred to herein shall refer to any type of patient-related data existing or generated during, before, or after the referral or medical disembark. The system of the present disclosure has the ability to connect and exchange data between multiple parties to coordinate care between shoreside entities, shoreside medical providers and a maritime vessel and/or vessels (i.e., a fleet of vessels), which may move locations or have various routes and/or destinations.

The system of the present disclosure will produce necessary medical and patient information that is automatically or manually exchanged between the maritime vessel and the necessary shoreside entities and shoreside medical providers in an efficient, compliant and secure way, according to various applied rules and logic. Some of this coordination may also include disembarkation or intake activities with an insurance company, medical assistance company, port agent or authority, government immigration or customs authority, health departments, medical transportation company, or standard transportation company, medical clinic or hospital and/or an overseeing organizational, management and/or operational body under which the system automates the required information exchange, reporting and documentation.

The system of the present disclosure has the ability to directly connect and exchange information with a medical provider through a software portal application of the system or third party software via application programming interface (API), electronic data interchange (EDI), health information exchange (HIE), direct connection, etc., that will allow the vessel and the medical provider and/or shoreside entities to coordinate various data interactions as identified throughout this disclosure. The system of the present disclosure has the ability for the provider to communicate information back directly with the organization or vessel via the provider portal application of the system or third party software via API, EDI, HIE, direct connection, or any other type of electronic communication. In addition to the above, the system of the present disclosure also has the ability for the vessel, shoreside medical provider or providers and/or shoreside entities to process payments with one another.

The system of the present disclosure provides for an entity to centrally track and manage patient cases from shoreside and provides the ability for the shoreside entities to track and manage the activities and medical disembarks or referrals, for seafaring vessels either owned, managed, serviced or operated, for the purposes of reporting and managing these cases. Advantageously, the system of the present disclosure allows vessels that are operating and/or navigating on bodies of water across the world to manage the complex situations that occur when a decision needs to be made as to whether someone onboard needs to leave the vessel due to an illness or injury often in a foreign or unfamiliar location far away from home or the point of embarkation. Identifying the appropriate medical care provider or providers under routine, urgent or emergency situations is complex. The complexity is further complicated by the need to coordinate and manage the requirements and needs of various entities prior to and upon arriving at a port location along with the need to also manage and coordinate the return of the patient to the vessel or the repatriation of the patient during or after medical treatment.

The problems solved by the system of the present disclosure cannot adequately be addressed by land-based healthcare systems that are not required to manage or coordinate the complexities that exist for vessels operating or moving on bodies of water across the world. Further, the system of the present disclosure has the ability to automatically calculate the need for repatriation of the patient to their home country or requested location based upon at least one or a combination of fitness for duty or fitness to sail, employment status, alternatives for care within their home country, patient or family consent, a calculated maximum medical improvement (MMI) status for the patient and/or contractual or legal requirements in accordance with their obligation to their employment as a crew member or a passenger on the vessel. The aforementioned list is not exhaustive and other factors may be considered.

The system of the present disclosure also automatically records and calculates the costs of the patient's case and applies the amount against a financial reserve or amount allocated or allotted per case or policy. Upon achieving certain thresholds, the system of the present disclosure manually or automatically notifies or makes available to the end user and/or respective parties (e.g., P&I club, payer, finance entity, human resource (HR) and/or staffing agency, ship management, shoreside medical provider, vessel, etc.) the status of the patient's case in order to take appropriate action, if needed. This information can then be made available directly to the parties, end user or servicer through the system, or third party software via, for example, API, EDI, HIE, direct connection, or other forms of electronic communication.

The figures illustrate the steps performed by four distinct entities: the user vessel, the user's shoreside system, the third party servicer, and the medical provider. The user vessel shall use the system of the present disclosure. The user's shoreside entity shall use the system of the present disclosure or access the system via API. The third party servicer and medical provider may also use the system's software portal or API or direct access (or other form of electronic communication) to access the system.

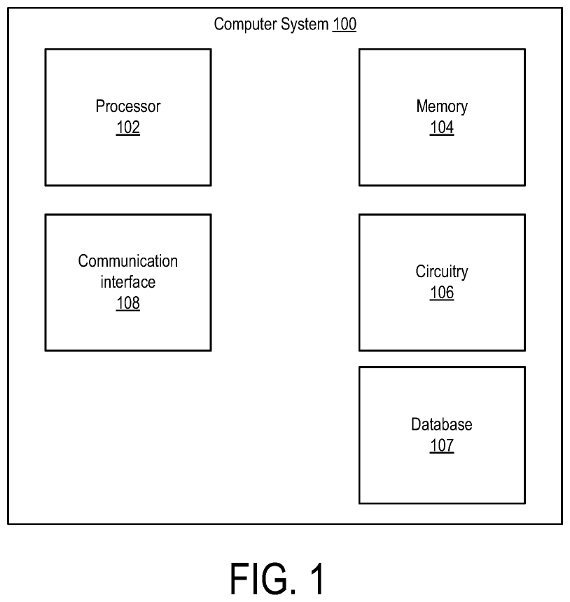

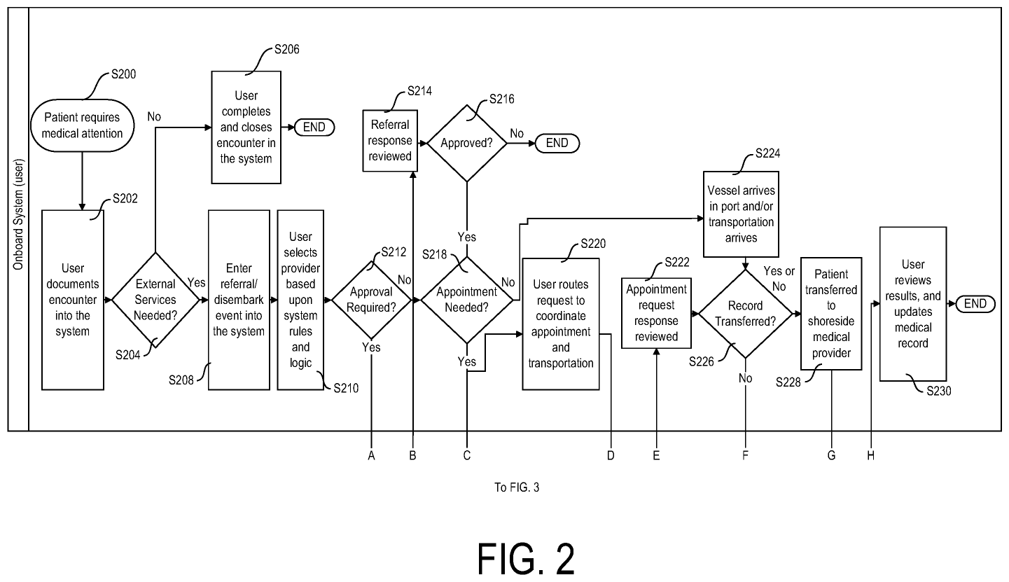

Referring now FIG. 1, an exemplary computer system 100 of the present disclosure is shown. Computer system 100 as defined herein shall be of the type used by the user in its onboard and shoreside systems, a crew portal computer system that allows a crew member to share their patient information with shoreside entities, the shoreside medical provider and/or the vessel, the computer system used by third party servicers (and their external systems) and the computer system used by shoreside medical providers (and their external systems) as explained below and in the flowcharts shown in FIG. 2 through FIG. 7.

In one embodiment, computer system 100 includes a traditional processor 102, a memory 104, processing circuitry 106, a database 107, and a communication interface 108. Processing circuitry 106 may include integrated circuitry for processing and/or control, e.g., one or more processors and/or processor cores and/or FPGAs (Field Programmable Gate Array) and/or ASICs (Application Specific Integrated Circuitry). The processing circuitry 106 may include and/or be connected to and/or be configured for accessing (e.g., writing to and/or reading from) the memory 104, which may comprise any kind of volatile and/or non-volatile memory, e.g., cache and/or buffer memory and/or RAM (Random Access Memory) and/or ROM (Read-Only Memory) and/or optical memory and/or EPROM (Erasable Programmable Read-Only Memory). Such memory 104 may be configured to store code executable by control circuitry and/or other data, e.g., data pertaining to communication, e.g., configuration and/or address data of nodes, etc.

The processing circuitry 106 may be configured to control any of the methods described herein and/or to cause such methods to be performed, e.g., by the processor 102. Corresponding instructions may be stored in the memory 104, which may be readable and/or readably connected to the processing circuitry 106. In other words, the processing circuitry 106 may include a controller, which may comprise a microprocessor and/or microcontroller and/or FPGA (Field-Programmable Gate Array) device and/or ASIC (Application Specific Integrated Circuit) device. It may be considered that the processing circuitry 106 includes or may be connected or connectable to memory 104, which may be configured to be accessible for reading and/or writing by the controller and/or processing circuitry 106. Database 107, which may be used to store related information, can be included as part of computer system 100 or may be a remote database in communication with computer system 100.

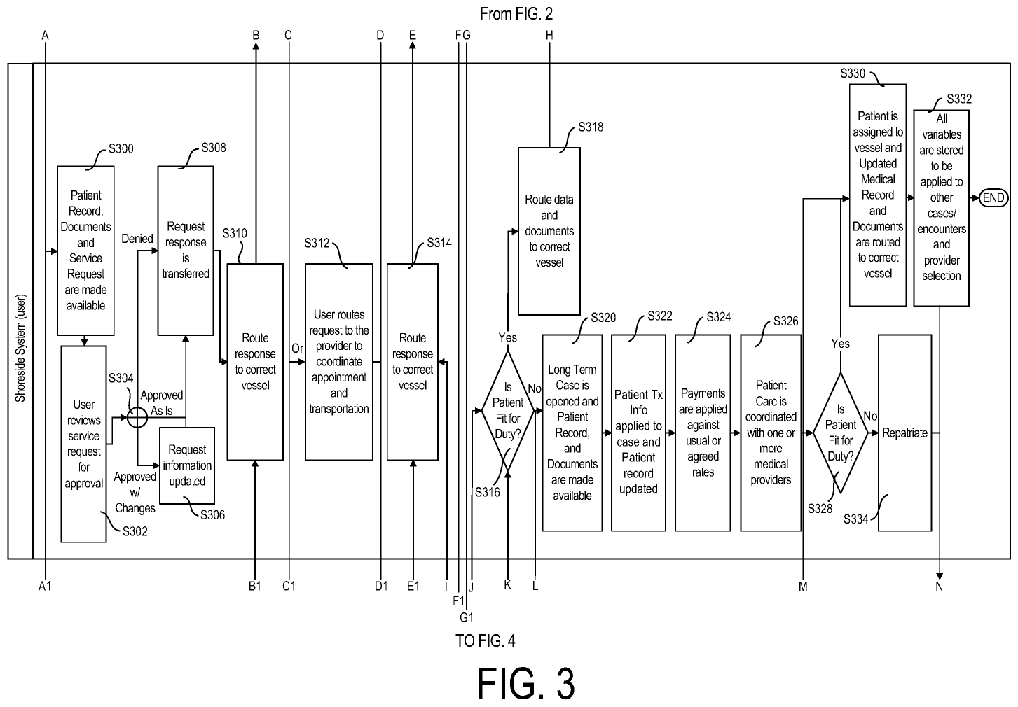

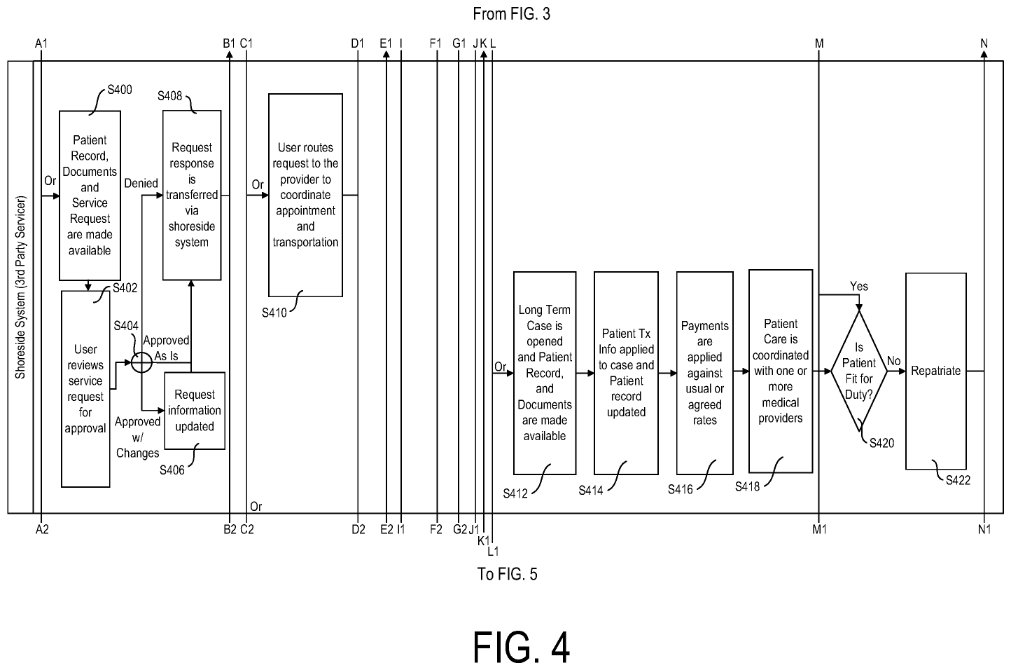

FIG. 2 illustrates a series of steps taken by the system onboard the vessel (“onboard system”) of the client or user in accordance with the principles of the present disclosure. The “onboard user” or “shoreside user” as that term is used herein shall refer to an individual who works on behalf of a vessel management entity. The vessel management entity can, for instance, manage what occurs onboard a vessel or a fleet of vessels. This may include, for example, obtaining and/or providing for medical care of passengers and crew members onboard the vessel. It should be noted that in one embodiment, the user is comprised of two entities—an onboard entity (having a computer system or “onboard system”) and a shoreside medical provider (having a computer system or “shoreside system”). The tasks performed by the user's onboard system in accordance with the principles of the present disclosure are discussed below and with regard to FIG. 2. The tasks performed by the user's shoreside system are discussed below and with regard to FIG. 3. It should also be noted that the user may share managerial responsibility with other managerial entities, such as third party servicers. The functions performed by the third party servicers are discussed below and with regard to FIGS. 3 and 4.

Referring now to FIG. 2, steps taken by the user's onboard system in accordance with the principles of the present disclosure are shown. In one embodiment, a crew member or passenger (e.g., “patient”) that is feeling unwell or is injured receives medical attention onboard a vessel. Thus, the patient can be assessed by an onboard nurse, physician, paramedic, medical care provider, or designated crew member. If medical attention is needed at step S200, then a patient “encounter” is created, and the patient's encounter is documented in the user's onboard system, at step S202. Then, the user, and/or the user's onboard computer system gathers and evaluates information from the patient, including, for example, patient medical history, patient personal information, patient vitals, primary medical complaint, diagnosis, review of the body systems, patient's physical examination results, results of onboard investigation or diagnostics, severity of the patient's condition, patient consent, etc. In one embodiment, the system will prompt or suggest whether the medical condition of a patient warrants a consult or services of a remote third party servicer or medical care provider (e.g., telemedicine, medical assistant services, etc.).

At this point, it is then determined if external shoreside services are needed, i.e., whether the patient needs a referral, or needs to disembark onshore to receive medical attention by an external source, at step S204. System 100 on board the vessel is able to record and manage medical information for the patient and, in one embodiment, processor 102 will prompt or suggest a particular level of care, e.g., whether the medical condition of a patient warrants removal of the patient to a shoreside medical provider, for medical care. This level of care that is suggested may vary and include, for example, standard, urgent or emergency care based upon certain configurations or applied rules or applying system driven intelligence or logic (e.g., static data, or machine learning/artificial intelligence (ML/AI)) against data variables. The data variables may be entered via user input, device input (i.e., diagnostic, wearables, etc.), a third party data source, or previously entered info such as, for example, patient history, vitals, chief complaint, diagnosis, geographic location, review of body systems, physical examination results, lab results, or other various medical or personal information.

Thus, for example, if it is determined that the patient's illness or injury does not require medical attention after all, or if the patient can be treated on board the vessel without the need to be transported off the vessel to an onshore medical provider, the patient's encounter is documented as closed in the system, at step S206. The user's onboard system, and, in some embodiments, all computer systems across the system, and its algorithms are updated to record that the patient's illness, and associated symptoms, do not rise to the requisite level of severity that warrants onshore medical attention. However, if the patient's medical condition is determined to be at a level that would require the patient to be treated off of the vessel at a shoreside medical provider, a referral/disembark event is entered and updated into the system, at step S208.

Processor 102 of computer system 100 then suggests a shoreside medical provider or a group of potential shoreside medical providers that can treat the patient, the suggestion based upon predetermined system rules and logic, at step S210. For example, processor 102 of computer system 100 may recommend the name of a suggested medical provider or present an option of providers to send the patient based upon various configurations or rules or applying system driven intelligence or logic, e.g., static data, and/or ML/AI, against data variables either entered, received via other data sources, interfaces, or calculations. These may include, for example, geographic location/region, GPS position, routes/itinerary, proximity, patient condition severity, diagnosis and medical specialty required, contractual arrangements, quality, customs/border/port control of the country where the patient is to be referred or to disembark, costs, third party lists, etc. The aforementioned list does not necessarily represent all of the data variables that may be considered in determining the shoreside medical provider or providers. The list of providers can be from a system-generated list, a manually-created list, or a third party source.

Because the vessel is on a body of water and constantly in motion, when a patient falls ill or is injured, the vessel may have the option to come ashore at various locations, i.e., various countries. This may include the country that the vessel had intended on arriving at or may also include a different country not on the vessel's itinerary. The system of the present disclosure considers such factors as the date, the vessel's location, its itinerary, its proximity and accessibility to the port. These factors may include but are not limited to such considerations as the ability of the port to support the type and size of the vessel (e.g., the depth of the water around the port, berth, and the overall navigability of the port), tides, size of the vessel, the country of origin of the vessel, the severity and diagnosis of the patient's malady, the medical providers' specialty areas, as well as customs/border control, port agents, port security, and port clearance factors for the various available countries where the vessel could possible conduct a referral or disembark. These factors are compared against a list of available medical providers. The aforementioned factors are non-limiting and some, all, or other factors may be considered. Other factors, are, for example, costs, quality of service, accessibility, agreements, language, location and transportation. Based upon this comparison, the most suitable medical provider is selected by processor 102.

It should be noted that instead of processor selecting the most suitable medical provider, instead, processor 102 can provide suggestions to the user or prompt the user. For example, the user can be provided with a listing of suitable and qualified shoreside medical providers, allowing the user to choose. Alternately, the processor 102 can provide prompts, thus, for example, providing the user with useful information/characteristics about each shoreside medical provider, again, in order to allow the user to ultimately make the selection of shoreside medical provider.

It is then determined if approval must first be obtained before the patient can be transported off of the vessel to an onshore medical provider, at step S212. For example, there may be standing rules that require additional approval from the user (for example, standard company procedures or regulatory), a shoreside entity, or a shoreside medical provider before the patient can be transported to the shoreside medical provider. For example, one third party servicer could be in charge of transporting the patient from the vessel to the medical provider when the vessel arrives shoreside. This servicer would need to first receive a request for approval to determine if they have an ambulance available at the time and location the patient needs to disembark. Similarly, the shoreside medical provider that is selected to treat the patient may need to first provide approval to treat the patient (e.g., guarantee of payment, specialists or equipment available at the location, etc.).

If approval is required, the user's onboard system sends an approval query to the relevant entity (shoreside vessel management entity, third party servicer or shoreside medical provider). This approval process is discussed below in greater detail with regard to FIG. 3 through FIG. 5. If approval was required, the user's onboard system receives and reviews the approval query response, at step S214. If, at step S216 is determined that no approval was obtained, the process terminates. If approval to refer or disembark the patient was granted, at step S216, or if no approval was needed, it is then determined, by processor 102 of the user's onboard computer system if an appointment with the shoreside medical provider is needed, at step S218. If an appointment is required, the user's onboard computer system routes an appointment request to the corresponding party (i.e., shoreside medical provider or one or more third party providers) to coordinate an appointment, at step S220.

If an appointment is required, an appointment request is sent and the appointment request response is received and reviewed, at step S222. The vessel arrives in port and/or transportation is arranged and the patient is transported to the shoreside medical provider, at step S224. The patient's record then needs to be transferred to the corresponding party that will be coordinating and/or treating the patient. Note that the patient's record can be transferred prior to the vessel arriving at port, during, or after arrival. Step S226 determines if the patient's record has been transferred to the selected shoreside medical provider. Once the patient's record is transferred, the patient is transferred to the selected shoreside medical provider, at step S228. In other embodiments, the transferring of the patient's record can occur during or after the patient arrives at the medical provider. During and after the time the patient is receiving medical care at the shoreside medical provider, the user's onboard system, which may be in the original or different location, receives the results of the performed medical care, and updates the patient's medical record, at step S230.

FIG. 3 illustrates steps taken by the user's shoreside system in accordance with the principles of the present disclosure. The user's shoreside system as that term is used herein is the application designed to provide shoreside entities (i.e., a vessel management entity, a third party servicer, etc.) with oversight and service capabilities (e.g., medical case management, referrals, disembarks, etc.) allowing for direct overview and collaboration with individual vessels and fleet operations. The shoreside system aggregates and synchronizes fleet information and metrics from all vessels and system components. This also includes the patient record, which follows the crew member on whichever vessel they are on, return to or board in the future. It should be noted that the shoreside system may be used/access by one or more shoreside entities.

Thus, the user, in some embodiments, may have the system on board the vessel, as well as the system shoreside, that is in communication with the system on the vessel, and also in communication with the system or computer systems of third party servicers, and shoreside medical providers. As described above, in step S212, approval may be required before the patient can leave the vessel for treatment by the selected medical provider. This approval may be required by the shoreside entities and/or the shoreside medical provider. If approval is required, the patient's record, documents and request for approval are made available through the system via data replication, at step S300. In other embodiments, for example, when the approving entity not using the host system, the approval may be made available through electronic communication other than data replication, for example, API, or other forms of electronic communication, etc., as described throughout this disclosure.

The user's shoreside system receives and reviews the service request for approval, at step S302. The service request is either approved as is, approved with changes, or denied, at step S304. If the service request is approved with changes, the request information is updated, at step S306, and a response to the approval request is prepared and transferred via data replication, at step S308. If the approving entity is not using the shoreside system, then the data arrives at the shoreside system first via API, or any of the other forms of electronic communication described herein and the data is then transferred to the appropriate vessel via data replication. The response to the service request, whether it has been approved as is, approved with changes, or denied, is routed to the user's onboard system via data replication utilizing system logic based upon the vessel manifest or the system of the vessel from which the request came, at step S310.

At step S312, if an appointment is required, the user's shoreside system routes an appointment request to the shoreside medical provider or third party servicer to coordinate an appointment and, if needed, to coordinate transportation of the patient. The transportation of the patient can be managed and coordinated by the shoreside medical provider, shoreside entities, onboard users, or other entities, for example, port agents, or government entities. After receiving the response to the appointment request from the medical provider or shoreside entities, the response is routed to the onboard system of the vessel from which the patient is to disembark from. The routing of this message to the correct vessel is done via data replication using system logic based on either the vessel manifest system or the system from where the request came from, at step S314.

After the patient has been examined and evaluated by the shoreside medical care provider, the shoreside medical provider determines the patient's fitness for duty or fitness to sail status, i.e., whether the patient is considered healthy enough to return to the vessel to work and/or sail, at step S316. In other embodiments, it is the user shoreside system, or a user at the shoreside entity that can determine if the patient is fit to sail or fit for duty. For example, after examination, the shoreside medical provider may not declare the status of the patient and the user shoreside system (or a user at the shoreside entity can do this manually) determines the status. In other embodiments, the shoreside medical provider provides a status but the user shoreside system overrides or changes the status. Note that in some embodiments, step S316 would not occur, i.e., there would be no need for a determination of the patient's fitness for duty or fitness to sail status if they underwent a minor medical procedure, such as, for example, a dental cleaning.

If the patient is fit for duty or fit to sail, patient-related data and documents are routed by the user's shoreside system to the onboard system of the vessel from which the patient came based via data replication using system logic based on either the vessel manifest system or the system from where the request came from, at step S318. In the instance where it is determined that the patient is not fit for duty, or not fit to sail, i.e., is not considered healthy enough to return to the vessel, a long term care case is opened for the patient and the patient's record and related documents are made available to the vessel and shoreside entities, at step S320. Thus, the patient's records are always accessible whether or not the patient has been found fit for duty/sail or not. In some embodiments, the patient's fitness for duty or sail is determined prior to the patient leaving the vessel. Thus, it is not always the shoreside medical provider that determines if a long term case is opened for the patient.

Patient information is continuously updated throughout the case, and calculations are made for administrative, financial, legal and operational factors (e.g., sick wage benefits, care plans and medically necessary treatments, invoices, payments, reserves, follow up, scheduling, communication, etc.), at step S322. Payments are calculated and applied to the provider or servicer and, when applicable, applied at the agreed upon rates, which can be stored in the system, at step S324. Patient information is stored, for example, in database 107 of the user's shoreside system, and patient care is coordinated with providers as needed, at step S326. At this time, if the patient needs further medical care, for example, referral to another specialist, the process repeats, e.g., authorization, if needed, is obtained, and an appointment, if needed, is made, etc.

At this point, it is again determined if the patient is fit for duty or fit to sail, at step S328. If so, the patient may be assigned back to the vessel (or to another vessel), and updated medical records and associated documents are routed to the onboard system of the correct vessel based on the manifest system or manual assignment via data replication, and also may be routed to the shoreside system which maintains a synchronized copy of the distributed and consolidated patient record and related information which is constantly updated, at step S330. Thus, the patient record is continuously updated, replicated, and synchronized by all entities and maintained as a single, updated patient record. The replication of the data to the shoreside system may be performed in any data replication format, i.e., continuous, scheduled, batch, etc. and made upon any change to the patient record and is not contingent upon whether or not the patient returns to a vessel.

All variables and algorithms are updated and stored in order for them to be applied to other cases and encounters and/or to improve shoreside medical provider selection, at step S332. If the patient is still not considered fit for duty or fit to sail and is therefore unable to return to the vessel, the patient either continues treatment with the shoreside medical provider, or is repatriated or transported to another shoreside medical provider based on a combination of factors such as but not limited to, a fitness for duty or fitness to sail status, medical needs or medical availability, employment status, alternatives for care within their home country, patient or family consent, a calculated maximum medical improvement (MMI) status for the patient and/or contractual or legal requirements, at step S334. This could mean the patient is repatriated to their own country of care or transported to another location.

FIG. 4 illustrates steps taken by a shoreside third party servicer in accordance with the principles of the present disclosure. In some embodiments, one or more third party servicers can share the management of the vessel along with the user's onboard and shoreside systems. For example, a third party servicer can be responsible for managing one or more vessels in a fleet, arranging for transportation of patients to shoreside medical providers, coordinating communication between the user's systems (onboard and shoreside) and the selected shoreside medical provider, managing and/or coordinating medical claims and/or payments with respective entities, providers, and/or payers, etc. The present disclosure includes embodiments where there are one or more third party servicers working together with the user's shoreside and onboard systems. The third party servicers can have managerial responsibilities that are distinct or that may overlap those responsibilities of the shoreside or onboard users.

The shoreside third party servicer receives the patient record, documents and service request for approvals that are made available through the system via data replication by direct connection, at step S400, reviews the service request, at step S402, either denies, accepts, or accepts with changes the service request for approval, at step S404, updates the request information (if necessary), at step S406, and transmits the response to the service request via the shoreside system, at step S408. Similar to step S312 of FIG. 3, if an appointment is required for the patient to be treated by the shoreside medical provider, the user's onboard system transfers the appointment request to the shoreside system used by the third party provider in order to coordinate the patient appointment and transportation of the patient to the shoreside medical provider, at step S410.

Steps S412 through S418 regarding when a patient is not fit for duty are the same as steps S320 through S326 in FIG. 3 and therefore these steps will not be repeated here. If the patient is still not considered fit for duty or fit to sail, at step S420, and is therefore unable to return to the vessel, the patient either continues treatment with the shoreside medical provider, is repatriated or transported to another shoreside medical provider based upon a combination of but not limited to, a fitness for duty or fitness to sail status, medical needs or medical availability, employment status, alternatives for care within their home country, patient or family consent, a calculated maximum medical improvement (MMI) status for the patient and/or contractual or legal requirements, at step S422. If the patient is considered fit for duty, the patient may be assigned back to the vessel (or to another vessel), and updated medical records and associated documents are routed to the onboard system of the correct vessel based on the manifest system or manual assignment via data replication, and also may be routed to the shoreside system which maintains a copy of the distributed and consolidated patient record and related information which is continuously updated, at step S330.

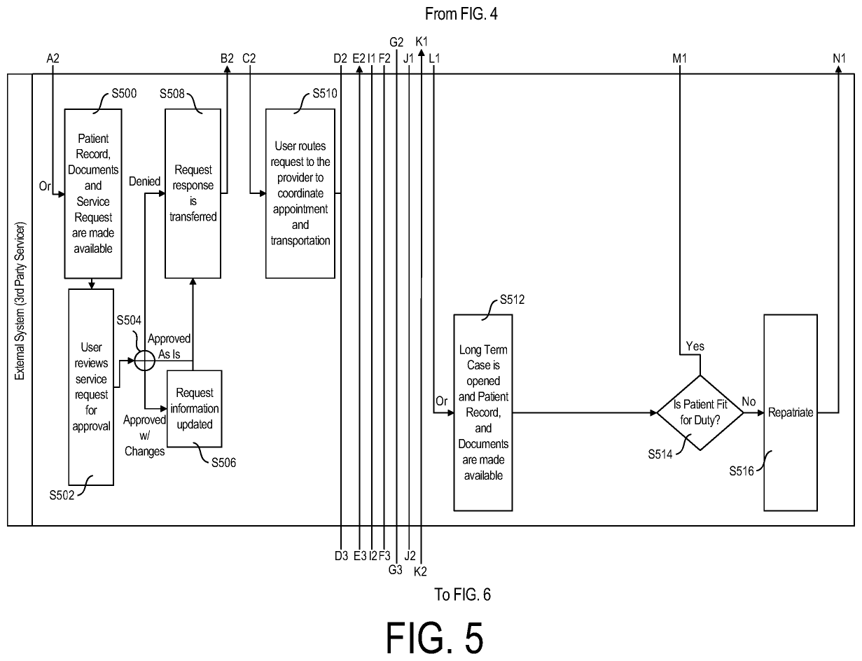

In one embodiment, the third party servicer has an external system that is separate from but in communication with the shoreside system of the shoreside vessel management entity. FIG. 5 illustrates steps taken by an external system of the third party servicer in accordance with the principles of the present disclosure. Steps S500 through S510 are similar to those performed by the shoreside system used by the third party servicer (steps S400 through S410) in FIG. 4 and will not be explained here in detail. The two differences are that in step S500, the external third party servicer system receives the patient record, documents and service request for approval that is made available by API, EDI, HIE, email, or any other forms of electronic communication. In step S400, this information was made available by direct connection via data replication. In step S508, the external third party servicer system transmits a response to the service request via API, EDI, HIE, email or other forms or electronic communication, as opposed to by use of the shoreside system in step S408.

Similar to step S412 in FIG. 4, if the patient is not considered fit for duty, a long term care case is opened for the patient and the patient's record and related documents made available, for example, via email or API, EDI, HIE, email, or other forms of electronic communication, at step S512. Finally, if the patient is still not considered fit for duty, at step S514, then the patient either continues treatment with the shoreside medical provider, is repatriated, or is transported to another shoreside medical provider, at step S516. If the patient is considered fit for duty, the patient may be assigned back to the vessel (or to another vessel), and updated medical records and associated documents are routed to the onboard system of the correct vessel based on the manifest system or manual assignment via data replication, and also may be routed to the shoreside system which maintains a copy of the distributed and consolidated patient record and related information which is updated via one or more data replication formats, e.g., continuous, batched, scheduled, etc. at step S330.

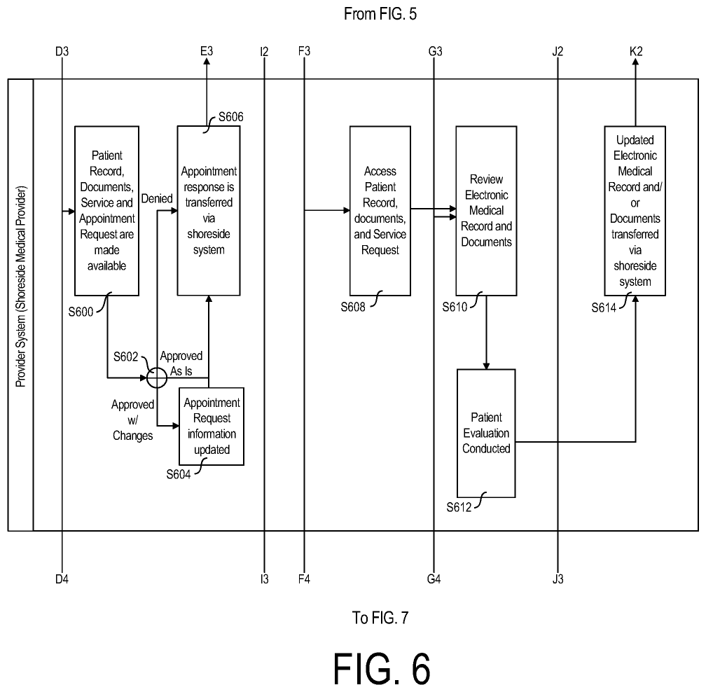

FIG. 6 illustrates steps taken by the system of the shoreside medical provider in accordance with the principles of the present disclosure. The shoreside medical provider is the entity selected in step S210. The shoreside medical provider's computer system receives the patient's record, associated documents, service request, and appointment request (if needed) through the system, at step S600. The provider's system can also be accessed/used by a third party servicer. The requests are then either approved by the shoreside medical provider as is, approved with changes, or denied, at step S602. If an appointment request was received and approved with changes, the revised appointment request is updated, at step S604. The appointment response, whether approved, approved with changes or denied, is transferred back to the vessel via data replication through the shoreside system, at step S606. The shoreside medical provider's system accesses the patient's record, documents, and service request through the provider system, at step S608, and reviews the electronic medical record for the patient along with any associated documents, at step S610. The patient is then evaluated by a qualified caregiver associated with the selected shoreside medical provider, at step S612. The provider system then updates the patient's electronic medical record, and the electronic medical record along with any associated documents are transferred back to the vessel via data replication through the shoreside system, at step S614.

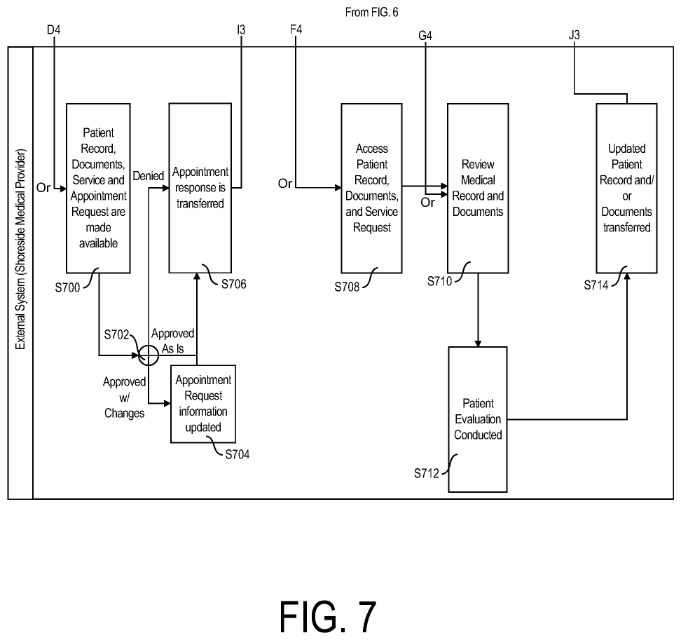

In one embodiment, the shoreside medical provider has an external computer system that is separate from but in communication with the shoreside system of the shoreside medical provider whose functions are shown in FIG. 6. FIG. 7 illustrates steps taken by an external system of the shoreside medical provider in accordance with the principles of the present disclosure. Again, the shoreside medical provider was selected in step S210 of FIG. 2. The external system of the shoreside medical provider receives the patient's record, associated documents, service request, and appointment request (if needed) through the provider system via an API, EDI, HIE, email or other forms of electronic communication (as described above), at step S700. The requests are then either approved as is, approved with changes, or denied, at step S702. If an appointment request was received and approved with changes, the revised appointment request is updated, at step S704. The appointment response, whether approved, approved with changes or denied, is transferred back to the shoreside system via API, EDI, HIE, email, or other forms of electronic communication, as described above, at step S706 and transferred back to the vessel via data replication.

The external system of the shoreside medical provider receives the patient's record, documents, and service request via API, EDI, HIE, email, or other forms of electronic communication, at step S708, and reviews the patient record along with any associated documents, at step S710. The patient is then evaluated by a qualified medical care giver associated with the selected shoreside medical provider, at step S712. It should be noted that the patient can be evaluated by a qualified medical care giver either at step S712 or step S612. If at step S612, the patient's record is accessed through the provider system, or if at step S712, the patient's record is received via API, EDI, HIE, email, or other electronic communication. Evaluation and/or treatment then follows. The provider system then updates the patient's electronic medical record, and the electronic medical record along with any associated documents are transferred back to the shoreside system via API, EDI, HIE, email, or other forms of electronic communication, at step S714, and transferred back to the vessel via data replication.

The system of the present disclosure produces the necessary medical, administrative, and patient information that is automatically exchanged between the maritime vessel and the necessary shoreside entities and shoreside medical providers in an efficient and secure way, according to various applied rules, functionality and logic. The system of the present disclosure coordinates the exchange of not only medical information but also automatically produces or exchanges information required for a crew member or passenger (“patient”) (i.e., a foreign national or citizen) to coordinate with and clear the vessel and authorities for disembark, such as with customs or port security.

Additionally, the system can also provide other administrative, financial, legal and operational information (i.e., personal information, details regarding required service, scheduling, costs, employment information, payment information, transportation information, governing agency information requirements, consents, etc.), required to facilitate services by a third party servicer provider (e.g., transport, escort, port agent, governmental agency, regulatory agency, public health agency, Customs, Coast Guard, etc.). Once it is determined that a referral or disembark of the patient will occur and the shoreside medical provider has been selected, the system will suggest and/or pre-populate relevant information for shoreside entities and/or shoreside medical providers that can be exchanged directly and electronically via a secure channel with the shoreside medical provider or shoreside entities. For example, the patient may need to be disembarked for a cardiac condition. The system will search for any cardiology, Customs or port security letter or notification formats in the system and present it to the user for exchange in order to address the medical information needs and any logistical or authority needs. For example, for Customs or the port authority needs, passport information, demographics, employment status, reason for entry into the country, medical needs, etc., may be needed. Logistical information that may be needed are, for example, the location of the provider, the type of transport needed, lodging, etc. Other authorities like public health agencies may need such information as the patient's health status, if they have any communicable diseases, etc. Finally, port agents which represent the shoreside vessel's organization management entity at the port may need information such as demographics, services required, location, payment information, transportation needs, lodging needs, etc.

In addition, onboard users can select a range of vital signs, lab results, encounter summaries, case summaries, documents, and/or administrative information (for example, citizenship, passport information, date of birth, payment terms, etc.) to be printed or sent electronically via a secure channel to the shoreside entities and/or shoreside medical providers. The shoreside medical provider can also send documents and/or structured data back in the same manner by the same or different modes of data transfer.

For clarity of explanation, in some instances the present technology may be presented as including individual functional blocks including functional blocks comprising devices, device components, steps or routines in a method embodied in software, or combinations of hardware and software.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims. Moreover, claim language reciting “at least one of” a set indicates that one member of the set or multiple members of the set satisfy the claim.

What is claimed is:

1. A vessel management system for facilitating patient care between a fleet comprised of at least one maritime vessel movable on a body of water and at least one shoreside medical provider, the system comprising:
a shoreside vessel management entity having a shoreside computer system including processing circuitry, the processing circuitry including a memory and a processor, the memory in communication with the processor, the memory having instructions that, when executed by the processor, configure the processor to aggregate and synchronize information and metrics from all vessels in the fleet including patient medical records; and
an onboard vessel management entity in communication with the shoreside vessel management entity, the onboard vessel management entity having an onboard computer system including processing circuitry, the processing circuitry including a memory and a processor, the memory in communication with the processor of the onboard vessel management entity, the memory having instructions that, when executed by the processor of the onboard vessel management entity, configure the processor of the onboard vessel management entity to:
upon determination that a patient on board the maritime vessel requires medical attention,
determine whether the patient should be referred to or medically disembarked to the at least one shoreside medical provider for medical care including one of routine, urgent or emergency care;
in response to determining, by the processor of the onboard vessel management entity, that the patient should be referred to or medically disembarked to the at least one shoreside medical provider,
determine an identity of potential ports that the vessel may disembark at, the determination based on depth of water around each port, and at least one of a type of and a severity of the patient's illness and at least one of a date, the vessel's location, the vessel's itinerary, and at least one of the vessel's proximity and accessibility to the port determined by applying system driven intelligence or logic against data variables including at least a global positioning system (GPS) or other positioning method to produce positioning data used to determine a relative distance between the vessel and the port;
utilizing at least the positioning data,
selecting from the potential ports a port which the vessel will disembark at including consideration of the port with a closest relative distance to the vessel based upon the positioning data,
in response to determining, by the processor of the onboard vessel management entity, the identity of the port which the vessel will disembark at,
identify a target shoreside medical provider from the at least one shoreside medical provider qualified to care for the patient; and
in response to identifying, by the processor of the onboard vessel management entity, the target shoreside medical provider,
coordinate care of the patient that is to be provided by the identified target shoreside medical provider, the coordination of care including coordinating exchange of any necessary information between the maritime vessel movable on a body of water and at least one of the identified target shoreside medical provider, one or more third party servicers, the shoreside vessel management entity, and one or more governing agencies, in order to facilitate the care of the patient, the exchange of necessary information occurring via a secure communication channel.

2. The vessel management system of claim 1, wherein the processor of the shoreside computer system is further configured to process a service request from the onboard vessel management for the patient to leave the vessel for treatment by the selected medical provider.

3. The vessel management system of claim 2, wherein the shoreside vessel management entity's response to the service request includes at least one of approving the service request, approving the service request with changes, and denying the service request, the response to the service request being routed to the onboard computer system via data replication utilizing system logic based upon the vessel manifest or the system of the vessel from which the request came.

4. The vessel management system of claim 1, wherein coordinating care of the patient that is to be provided by the identified target shoreside medical provider is performed by the shoreside vessel management entity.

5. The vessel management system of claim 1, wherein the processor of the shoreside computer system is further configured to determine if the patient is at least one of fit to sail and fit for duty.

6. The vessel management system of claim 1, wherein if the shoreside medical provider makes a determination that the patient is or is not fit for sail or fit for duty, the processor of the shoreside computer system is further configured to override or change the status of the shoreside medical provider's determination.

7. The vessel management system of claim 5, wherein if the patient is fit for duty or fit to sail, the processor of the shoreside computer system is further configured to route patient-related data and documents to the onboard vessel management entity.

8. The vessel management system of claim 1, wherein determining whether the patient should be referred to or medically disembarked to the at least one shore side medical provider comprises consideration of at least one of patient medical history, patient personal information, patient vitals, primary medical complaint, diagnosis, patient physical examination results, results of onboard investigation or diagnostics, availability of medical care of qualified personnel onboard the maritime vessel, severity of patient's condition, patient consent and geographic location of the maritime vessel.

9. The vessel management system of claim 1, wherein identifying a target shoreside medical provider from the at least one shoreside medical provider qualified to care for the patient comprises consideration of at least one of location of the maritime vessel, proximity and access of the maritime vessel to the at least one shoreside medical provider, severity of the patient's condition, diagnosis, medical specialty required, contractual arrangements, quality of medical care at the at least one shoreside medical provider, customs of the country where the patient is to disembark, and costs.

10. The vessel management system of claim 1, where coordinating the exchange of the necessary information includes consideration of at least one of, the patient's medical information, patient status, scheduling information, costs, employment information, payment information, transportation information, governing agency requirements, consent, and fit-for-duty or fit to sail status prior to, during, or after the referral or medical disembark.

11. The vessel management system of claim 1, wherein after care is provided by the target shoreside medical provider, further comprising determining, by at least the processor of the shoreside computer system, if the patient is to continue treatment with the shoreside medical provider, is to be repatriated, or is to be transported to another shoreside medical provider.

12. The vessel management system of claim 11, where determining if the patient is to continue treatment with the shoreside medical provider, is to be repatriated, or is to be transported to another shoreside medical provider depends at least upon the patient's fitness for duty, the patient's fitness to sail status, medical needs, medical availability, employment status, alternatives for care within the patient's home country, patient or family consent, a calculated maximum medical improvement (MMI) status for the patient, and contractual or legal requirements.

13. The vessel management system of claim 1, wherein the processor of the shoreside computer system is further configured to assign the patient to the vessel if the patient is fit for duty or fit to sail, and update the patient record after care has been provided.

14. The vessel management system of claim 1, wherein the processor of the onboard computer system is further configured to determine a required level of care for the patient comprising applying at least one of static data, machine learning, or artificial intelligence against data variables.

15. The vessel management system of claim 1, wherein determining the identity of the port that the vessel may disembark at is further based on berth at the port that the vessel will disembark at.

16. The vessel management system of claim 1, wherein determining the identity of the port that the vessel may disembark at is further based on tide.

17. The vessel management system of claim 1, wherein determining the identity of the port that the vessel may disembark at is further based on port security.

18. The vessel management system of claim 1, wherein determining the identity of the port that the vessel may disembark at is further based on a country of origin of the vessel.

19. The vessel management system of claim 1, wherein determining the identity of the port that the vessel may disembark at is further based on depth of water.

20. The vessel management system of claim 1, wherein determining the identity of the port that the vessel may disembark at is further based on available port services.

21. A vessel management system for facilitating patient care between a fleet comprised of at least one maritime vessel movable on a body of water and at least one shoreside medical provider, the system comprising:
a shoreside vessel management entity having a shoreside computer system including processing circuitry, the processing circuitry including a memory and a processor, the memory in communication with the processor, the memory having instructions that, when executed by the processor, configure the processor to aggregate and synchronize information and metrics from all vessels in the fleet including patient medical records; and
an onboard vessel management entity in communication with the shoreside vessel management entity, the onboard vessel management entity having an onboard computer system including processing circuitry, the processing circuitry including a memory and a processor, the memory in communication with the processor of the onboard vessel management entity, the memory having instructions that, when executed by the processor of the onboard vessel management entity, configure the processor of the onboard vessel management entity to:
upon determination that a patient on board the maritime vessel requires medical attention,
determine whether the patient should be referred to or medically disembarked to the at least one shoreside medical provider for medical care including one of routine, urgent or emergency care;
in response to determining, by the processor of the onboard vessel management entity, that the patient should be referred to or medically disembarked to the at least one shoreside medical provider,
determine an identity of potential ports that the vessel may disembark at, the determination based on depth of water around each port, and at least one of a type of and a severity of the patient's illness and at least one of a date, the vessel's location, the vessel's itinerary, and at least one of the vessel's proximity and accessibility to the port determined by applying system driven intelligence or logic against data variables including at least a global positioning system (GPS) or other positioning method to produce positioning data used to determine a relative distance between the vessel and the port;
utilizing at least the positioning data,
selecting from the potential ports a port which the vessel will disembark at including consideration of the port with a closest relative distance to the vessel based upon the positioning data; and
in response to determining, by the processor of the onboard vessel management entity, the identity of the port which the vessel will disembark at,
identify a target shoreside medical provider from the at least one shoreside medical provider qualified to care for the patient.
